(19) United States
(12) Patent Application Publication
(10) Pub. No.: US 2022/0378338 A1
(43) Pub. Date: Dec. 1, 2022

(54) METERING DEVICE FOR BLOOD GLUCOSE CONCENTRATION BASED ON MICROWAVE SIGNALS

(71) Applicants: Jorge CASTIÑEIRA MOREIRA, Mar del Plata, Provincia de Buenos Aires (AR); CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE MAR DEL PLATA, Mar del Plata, Provincia de Buenos Aires (AR)

(72) Inventors: Jorge CASTIÑEIRA MOREIRA, Mar del Plata, Provincia de Buenos Aires (AR); Juan Carlos BONADERO, Mar del Plata, Provincia de Buenos Aires (AR); Alejandro José URIZ, Mar del Plata, Provincia de Buenos Aires (AR); Iván Exequiel GELOSI, Mar del Plata, Provincia de Buenos Aires (AR); Ramiro AVALOS RIBAS, Mar del Plata, Provincia de Buenos Aires (AR); Leonardo Oscar COPPOLILLO, Mar del Plata, Provincia de Buenos Aires (AR)

(21) Appl. No.: 17/753,781

(22) PCT Filed: Sep. 10, 2020
(86) PCT No.: PCT/IB2020/058423

§ 371 Date: Mar. 14, 2022

(30) Foreign Application Priority Data
Sep. 13, 2019 (AR) P20190102600

(51) Int. Cl. A61B 5/145; A61B 5/0507; A61B 5/00

(52) U.S. Cl. CPC A61B 5/14532; A61B 5/0507; A61B 5/6826; A61B 5/6844; A61B 2562/0228; A61B 2562/164

(57) ABSTRACT

Non-invasive measuring device for measuring blood glucose concentrations on a user's finger, based on microwave signals sensitive to differences in electrical permittivity caused by changes in blood glucose concentration, comprising a rectangular cross-sectional hollow prismatic resonant cavity, which is filled with a dielectric material, an antenna and a mold; a signal-generating means capable of generating microwave signals connected to the antenna through a signal-differentiating means; a signal-detecting means connected to the antenna through the signal-differentiating means; at least one means of control, connected to the signal-detecting means and to the signal-generating means, which controls signal generation and signal reception from the signal-detecting means.

### FIELD OF THE INVENTION

The present invention relates to the area of non-invasive clinical analysis, in particular those performed by non-invasive devices that can be used by the patients themselves who suffer a disease. In particular the clinical analysis carried out by the device of the present invention is the determination of glucose concentration in blood (glycaemia).

### BACKGROUND OF THE INVENTION

Over the past 50 years, the use of microwave-based instruments has been increasing progressively in a very broad set of applications. The capability of these instruments to non-destructively measure parameters within a volume makes them ideal for measuring in contexts where direct contact with the sample is not possible. This feature of microwave sensors suggests the convenience of their use for non-invasive measurements of human physiological parameters. One of these parameters is blood glucose concentration, the determination of which is an increasing need for monitoring patients with diabetes.

A person's glucose levels vary along a journey. Baseline blood glucose concentration values for a healthy person are between 70 mg/dL and 110 mg/dL after an extended fasting period. After food intake, concentrations could rise above 180 mg/dL, for short periods of time in healthy people. The World Health Organization typifies that people who potentially suffer from diabetes are those whose glycaemia values remain above 140 mg/dL after two hours of food intake. Depending on diabetes severity, blood glucose concentration should be measured at least 4 times per day. This measurement is performed in a blood sample obtained from a finger puncture with a specific, sharp object and pressure application until a drop of blood is obtained and placed on a measuring tape for use in a glucometer.

Given the extent of diabetes (number of cases worldwide), there are significant scientific and technological efforts to develop noninvasive methods and devices for measuring blood glucose concentration. In particular, methods based on the use of microwave signals are based on the capability of microwaves to pass through the components of human or animal biological tissue so as to be able to measure the fluid inside. If the object under analysis is a finger composed of skin, muscle, and bone, for example, a device that sends microwave signals to that object will be able to pass through the finger components and interact with the blood inside.

One of the fundamental properties of microwaves is their sensitivity to the electrical permittivity of materials. Permittivity (also called dielectric constant) is a physical constant that describes how an electric field affects and is affected by a medium. In this case, the significant feature is that changes in blood glucose content modify the electrical permittivity values of the blood. Thus, given an incident microwave signal, the reflected signal will be modified according to the electrical permittivity values of the fluid under study. These measurements then allow, after a proper calibration, to derive glucose values present in a blood sample.

When constructing a microwave device for measuring blood glucose concentration it is necessary to take into consideration several issues: development of a resonant cavity where a tissue under study is to be placed, the cavity confining inside the electric field; existence of a model accounting for the components of the body part and/or tissue where blood glucose changes are to be measured; and the microwave frequency range that allows the highest glucose sensitivity as well as optimizing efficiency, portability and costs. The information to be measured for characterizing blood glucose concentration will be on the whole response curve to incident signals due to a frequency scan where the resonance frequency will the variable to be optimized.

In 2005 Eric C. Green submitted and obtained approval of his master's thesis entitled Design of a Microwave Sensor for Non-Invasive Determination of Blood-Glucose Concentration—Department of Electrical Engineering and Computer Science at Baylor University. In his work, a microwave sensor was developed to measure blood glucose in a non-invasive manner based on a single-spiral microstrip resonant sensor.

Patent application US 2008/0319285 assigned to Ferlin Medical Ltd. filed on Jul. 6, 2006 and published on Dec. 25, 2008, discloses a device and method for measuring constituents within the structure of a biological tissue. In particular, said device and method are applicable for the detection of blood glucose.

The device in US′285 includes a microwave signal generation source capable to generate a microwave frequency range, a first antenna connected to the signal source with the capability to transmit at least a portion of the microwave signal to the tissue under analysis. A second antenna intended to receive at least a portion of the microwave energy transmitted through the tissue structure, a signal processor the function of which is to obtain the resonance frequency of the received microwave signal and a processor intended to provide an output with the concentration of existing constituents within the structure of the biological tissue according to the measured resonance frequency.

The antennas used in US′285 are two flat patch- or microstrip-type antennas. Antennas of this type couple their electromagnetic fields with the tissues immediately in contact or very close to them, with the consequent problem of the tissue position in relation to the antennas, which are extremely sensitive to said arrangement.

Patent application US 2012/0150000 (Ahmed Al-Shamma'a) filed on May 11, 2010, and published on Jun. 14, 2012 discloses a non-invasive blood constituent monitoring device in a subject. In particular, this device is applicable for blood glucose detection. The application concerns non-invasive monitoring of the concentration of some constituent of human or animal blood. In the preferred embodiment, the device comprises a circuit that provides an alternating current at microwave frequencies. This frequency is adjustable. The device has a sensor adapted for placement in the vicinity of a human or animal body. This sensor is connected to the mentioned circuit to receive the mentioned alternating current and adapted to send the mentioned signal to the corresponding body part. A detector is provided that receives the transmitted and/or reflected signal by the sensor. The properties of the detected signal depend on the concentration of the aforementioned blood constituent.

Patent application US′000 presents, in the preferred embodiment, a microstrip resonator-type structure in the form of a ring. As mentioned above for patent application US′285, structures of this type couple their electromagnetic fields with adjacent or very close tissues, with the consequent problem of the tissue position in relation to the sensor, the sensor being extremely sensitive to said arrangement.

Patent application GB 2428093 assigned to Microoncology Ltd. filed on Jul. 6, 2005, and published on Jan. 17, 2007, discloses a non-invasive blood constituent monitoring system in a subject. In particular it is applicable to blood glucose detection. It is a non-invasive instrument for obtaining information from biological systems using energy sources in the electromagnetic spectrum region. The measurement of the phase and/or magnitude of the signal changes is used to measure concentrations of several constituents present in these biological systems. More specifically, this invention can be used for non-invasive measurement of blood glucose levels.

The instrument of application GB′093 comprises a power source, an antenna array, and a mechanical system to monitor portions of transmitted and reflected energy, a frequency, phase and magnitude detection device, and a signal processor and an interface to produce an output with a friendly format.

Patent application GB′093 is broader version encompassing patent application US′285, so it faces the same disadvantages.

Patent application AR 104766 A1 assigned to the Consejo Nacional de Investigaciones Cientificas y Técnicas (National Council for Scientific and Technical Research) filed on Jul. 21, 2015, and published on Aug. 16, 2017, discloses a sensor device based on an open coplanar resonator that while it may have a satisfactory response, it has also a considerable sensitivity to the position of the finger leading to a poor repeatability of the results obtained from the measurements. In addition, the open arrangement of the resonator is susceptible to electromagnetic fields present in the environment and, therefore, to interference from external devices, communication signals, line noise, etc. In addition, the coplanar resonator also couples its electromagnetic fields with the tissues immediately in contact or very close to it. This introduces the problem of the position of the tissue in relation to the sensor, which is extremely sensitive to that arrangement. Finally, as in patent application AR′766 there is contact between the surfaces of the open coplanar resonator and the tissue, another problematic factor is the strength of that contact, since the pressure exerted on the biological tissue affects its shape, which in turn affects measurements.

Given the importance of glucose level control in patients suffering from diabetes, there is a permanent need for non-invasive devices, which are simple-handling for the user and provide results with the precision needed for patient care, overcoming the persistent inconveniences cited above.

### SUMMARY OF THE INVENTION

The present invention comes to satisfy this need by providing a measuring device that works similarly to a transducer, which is a device capable of transforming or converting a certain manifestation of input energy, into a different one at the output, but of very small values in relative terms with respect to a generator.

The measuring device of the present invention allows the measurement of changes in the electrical properties of materials, in this case, applied to glycaemia measurement. To this end, it makes use of a resonant cavity which is a microwave resonator that allows energy to be stored in the form of electromagnetic waves, which are affected by the dielectric characteristics of the materials present in the medium that is passed through by the microwaves. These effects on the waves can be observed from the source generating the electromagnetic wave, monitoring the amount of energy reflected towards the source. By inserting, for example, a finger into the resonator, the finger will disturb the waves, this disturbance will be measurable, and will depend on the blood glucose concentration.

It should be noted that a resonator is a structure with at least a natural frequency of oscillation or resonance. When the resonator resonates, its energy is alternately converted from one type to another, electrical and magnetic, depending on the frequency of that oscillation. If more energy is delivered to the resonator, it will be stored within it. Therefore, the resonator is capable to store energy in constant conversion.

In this way, in a resonant cavity, electromagnetic waves travel back and forth between reflective discontinuities, resulting in a stationary wave pattern. Resonant cavities usually have more than one resonance frequency. The values of these frequencies depend on the shape of the structure, its size, and the dielectric and/or magnetic properties of the mediums by which the waves travel.

Changes in glucose concentration can be detected by a microwave sensor, such as a resonant cavity, as the dielectric composition of a biological tissue sample alters the distribution of electromagnetic fields present in the sensor. In addition, the dielectric properties of blood and subcutaneous tissues change due to changes in plasma glucose levels to a much greater extent than variations in other compounds present therein.

Based on the above, it is possible to design a non-invasive sensor for measuring glycaemia levels. This measurement is indirectly performed because blood glucose levels produce a variation in dielectric properties of the blood, and by means of a microwave resonator it is possible to measure these variations.

Consequently, it is an object of the present invention a non-invasive measuring device to measure blood glucose concentrations in a user's finger, based on microwave signals sensitive to differences in electrical permittivity caused by changes in blood glucose concentration, the device comprising:
a hollow prismatic resonant cavity, with a width, height and length, with a rectangular cross-section defined by the width and height of the resonant cavity, being the longitudinal central axis of the resonant cavity in a horizontal position, where the resonant cavity is filled with a dielectric material, and where the resonant cavity comprises:
   two longitudinally opposite faces defined by the width and the height of the resonant cavity that define longitudinal ends of the resonant cavity, two laterally opposite faces defined by the length and the height of the resonant cavity, and two vertically opposite faces defined by the width and the length of the resonant cavity;
   an antenna inside the resonant cavity, capable of transmitting a microwave signal to the blood, being the antenna inserted into a hole located on one of the laterally opposite or vertically opposite faces;
   a mold for location and hold of the user's finger inside the resonant cavity, being the mold inserted into a hole located on at least one of the laterally opposite faces or into a hole located on at least one of the vertically opposite faces;
a signal-generating means capable of generating microwave signals connected to the antenna through a signal-differentiating means;
a signal-detecting means connected to the antenna through the signal-differentiating means; and
at least one means of control, connected to the signal-generating means and to the signal-detecting means, which controls the generation of signals from the signal-generating means and the reception of signals from the signal-detecting means.

In an embodiment of the present invention the resonant cavity is obtained from a metallic hollow prismatic profile, preferably made of bronze.

In an embodiment of the present invention the signal-generating means generates microwaves signals in the range of 0.5-3 GHz.

In an embodiment of the present invention the dielectric material is epoxy resin that presents a relative dielectric constant ER approximately equal to 4.3.

In an embodiment of the present invention the signal-differentiating means is selected from the group comprising a bidirectional coupler, a directional coupler and a circulator.

In an embodiment of the present invention the at least one means of control comprises a means of control connected to the signal-detecting means and that controls the reception of signals from the signal-detecting means, and a means of control connected to the signal-generating means and which controls the generation of signals.

In a preferred embodiment of the present invention the at least one means of control is selected from the group comprising a microprocessor and a programmable gate array.

In an embodiment of the present invention both laterally opposite faces each comprises a hole for insertion of the mold so that both holes are concentric to each other.

In another embodiment of the present invention both vertically opposite faces each comprises a hole for insertion of the mold so that both holes are concentric to each other.

In a preferred embodiment of the present invention the antenna and the mold are parallel to each other.

In a preferred embodiment of the present invention the antenna and the mold are located on the same face and aligned with the longitudinal center axis of the resonant cavity.

In a preferred embodiment of the present invention the resonant cavity has a width equal to twice its height.

In a more preferred embodiment of the present invention the resonant cavity has a width of 50 mm, a height of 25 mm and a length of 110 mm.

In an even more preferred embodiment of the present invention the optimal location of the mold is at a distance of 21 mm from one end of the resonant cavity and the optimal location of the antenna is at a distance of 22 mm from the other end of the resonant cavity.

In a preferred embodiment of the present invention the antenna is at a distance equal to a quarter of the length of the resonant cavity relative to one of its ends and the mold is at a distance equal to one quarter of the length of the resonant cavity from the other end, so that the antenna and the mold are separated from each other by a distance equal to half the length of the resonant cavity.

In a more preferred embodiment of the present invention the antenna is at a distance approximately equal to one fifth of the length of the resonant cavity relative to one end and the mold is at a distance approximately equal to one fifth of the length of the resonant cavity from the other end, so that the antenna and the mold are separated from each other by a distance approximately equal to three fifths of the length of the resonant cavity.

In a preferred embodiment of the present invention the position of the mold with respect to the position of the antenna is of half wavelength of the used microwave.

In a more preferred embodiment of the present invention the antenna is located at a distance of a quarter of the wavelength of the microwave used from one end of the resonant cavity and the mold is located at a distance from the antenna of half wavelength of the used microwave.

In an embodiment of the present invention the mold for location and hold of the finger is made of rigid plastic material.

In a more preferred embodiment of the present invention the measuring device comprises an additional mold of flexible plastic material for holding the user's finger, located inside the rigid plastic material mold. Preferably, the rigid plastic material mold has an inner threading for fastening the flexible plastic material mold.

In a more preferred embodiment of the present invention the flexible plastic material mold comprises an optical distance sensor to control that the level of finger penetration within the mold is suitable for measuring.

In an embodiment of the present invention the measuring device measures the reflection coefficient of the input of the resonant cavity (“parameter S11”) instead of the gain (“parameter 521”).

### BRIEF DESCRIPTION OF FIGURES

FIG. 1 is a top isometric perspective view of the hollow prismatic resonant cavity with rectangular cross-section of the present invention with an inserted mold.

FIG. 2 is a side view of the rectangular cross-sectional hollow prismatic resonant cavity rectangular cross-section of FIG. 1 with inserted mold and antenna.

FIG. 3 is a cross-sectional side view of the resonant cavity of FIG. 2.

FIG. 4 is cross-sectional side view of the resonant cavity of FIG. 2 with the addition of an additional mold.

FIG. 5 shows a block diagram that schematically represents how the different components that make up the measuring device of the present invention interact with each other.

FIGS. 6A to 6E show the frequency response of the measuring device of the present invention after a frequency scan using a spectrum analyzer.

FIGS. 7A to 7J show clinical results in the form of graphs showing the relationship between blood glucose concentration and resonance frequency over time for different volunteers after ingesting glucose powder dissolved in water.

FIGS. 8A and 8B show graphs of blood glucose vs. frequency that show how the metering device of the present invention improves glycaemia prediction through a self-training neural network, adjusting more precisely to the measurements obtained by an invasive glucometer.

FIG. 9 shows a graph of glycaemia vs. frequency that shows a calibration curve of the measuring device of the present invention for a volunteer with diabetes.

FIGS. 10A and 10B show the response in frequency using a device of the prior art and using the measuring device of the present invention.

### DETAILED DESCRIPTION OF THE INVENTION

Given the extent of diabetes (number of cases worldwide), there are significant scientific and technological efforts to develop noninvasive methods and devices to measure blood glucose. In particular, the method based on the use of microwave signals is based on the capability of microwaves to pass through the components of a finger such as skin, muscle and bone, for example, and thus interact with the blood present inside. A property of all materials, which is electrical permittivity, determines how the tissue behaves when interacting with microwaves, thus also affecting that electromagnetic wave. In this case, the significant feature is that changes in blood glucose content modify the electrical permittivity values of the fluid. Thus, given an incident microwave signal, the reflected signal will be modified according to the electrical permittivity values of the fluid under study. Therefore, the introduction of a finger, as described below, will modify the response in reflected frequency or signal in the measuring device of the present invention, so that by measuring the reflected signal and, from an appropriate calibration, the values of glucose present in blood can be obtained. More specifically, by means of graphs obtained through clinical tests on healthy volunteers and on volunteers with diabetes who used the measuring device of the present invention, as described below, when glucose concentration increases, the resonance frequency of the measuring device decreases and when the glucose concentration decreases the resonance frequency increases.

For the purposes of the present invention, the terms “resonant cavity” and “resonator” are used interchangeably. Similarly, the terms “microwave” and “wave” are used interchangeably to refer to electromagnetic waves in the microwave range, and the expression “sensor” will always refer to the resonant cavity unless the context clearly indicates otherwise. In addition, the terms “parameter S11”, “S11 response” and “frequency response” or derivations thereof all refer to the reflected signal or wave in the resonant cavity which separates from the signal or wave incident to that resonant cavity.

In each one of the figures the same numerical references are used for each element of the device of the invention.

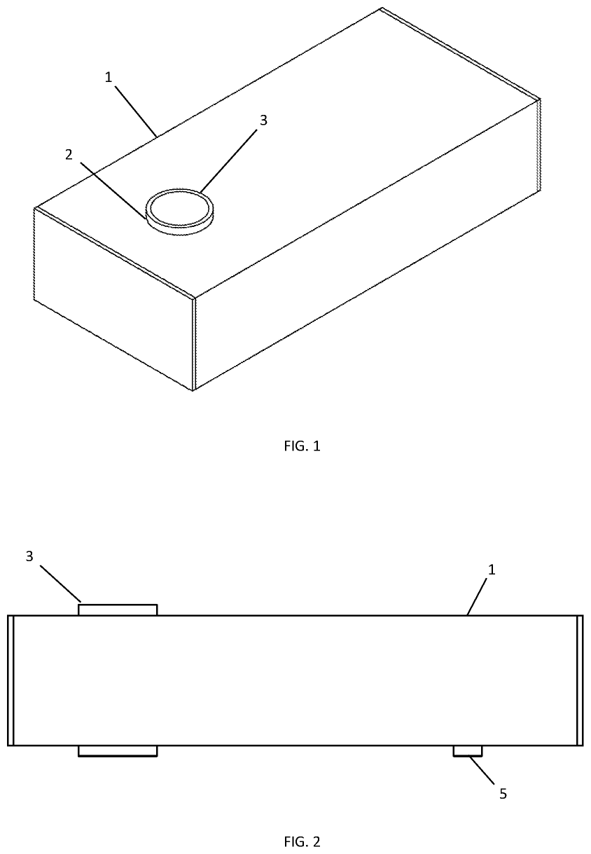

With reference to FIG. 1, it shows a top isometric perspective view of an embodiment of a hollow prismatic resonant cavity 1 with rectangular cross-section, filled with a dielectric material (not shown) of the device of the present invention, comprising a mold 3 for the location and hold of a finger, where mold 3 is inserted through a hole 2 on the upper face of the resonant cavity 1. In a preferred embodiment, resonant cavity 1 also comprises a hole 2′ (not shown) on the lower face so that holes 2 and 2′ are concentric to each other and that when mold 3 is inserted for finger placement and hold, said mold 3 passes through both holes 2 and 2′ and is fixed to them by, for example, a snap fit. The use of holes 2 and 2′ allows a more mechanically accurate fixation of mold 3, a calibration more suitable for different users and simplifies the filling process with the dielectric material. Such resonant cavity 1 can be easily obtained from a metallic hollow prismatic profile, preferably from bronze, with rectangular cross-section which is then closed by placing caps.

With reference to FIG. 2 now, it shows a side view of resonant cavity 1 of FIG. 1 comprising mold 3 and an antenna 5. It should be noted that while the inside of resonant cavity 1 can be left unfilled, that is, containing only air, the electrical characteristics of this medium require that the resonant cavity have very large dimensions, which makes low the volumetric participation of the human finger, and therefore the changes in microwave frequency produced by the finger are small, and would actually be produced mainly by the remaining structure of the resonant cavity.

It should be noted that in resonant cavities it is possible to input an electromagnetic signal that, under certain circumstances established by the geometry of the structure and the constituting materials, keeps the inner energy of the resonant cavity in resonance modes that occur at certain frequencies and are possible to be measured by using the appropriate instrument. When this electromagnetic signal is input, a sustained exchange of electrical to magnetic energy occurs and vice versa, which, at resonance frequencies, involves a maximum amount of energy in the resonant cavity.

These resonances depend on the dimensions and geometry of the resonant cavity, as well as the material that fills the same. The operating principle of the non-invasive glucose measuring device, as described below, is to make a human finger part of the inner structure of the resonant cavity, by inserting it at the appropriate point that maximizes the effect of the electric field on the finger. In this way, acted by an electric field that is distributed with high intensity on the finger, it is achieved that changes in the dielectric constant that result from changes in blood glucose level, have a significant impact on the resonator, changing both its frequency response and its resonance frequency. Analyzing and processing frequency response variables such as resonance frequency, amplitude of the reflected signal in the resonance frequency, and reflected amounts in frequencies in the resonance frequency environment (bandwidth), the level of present blood glucose can be determined. The mentioned variables are by way of example, and should not be interpreted as the only data to be used for blood glucose determination, being possible to use other simple measurement data from analysis of the frequency response in order to improve the obtained results.

In rectangular resonant cavities, resonant modes or resonance modes are designated TE_{n,m,l}, where integers “n”, “m” y “1” refer to the number of electric field maximums in a stationary wave pattern in each of the coordinate directions, respectively. As seen below, f_{r;n,m,l} refers to the resonant frequency for a given mode and the way to obtain it for a rectangular resonant cavity is given by the following equation

$${f}_{r ; n , m , l} = \frac{v}{2} \sqrt{{( \frac{n}{a} )}^{2} + {( \frac{m}{b} )}^{2} + {( \frac{l}{L} )}^{2}} \qquad ( 1 )$$

Where a and b are the width and height of the resonant cavity (in its transverse dimensions) and L is its length. Respectively, n, m, and l refer to Cartesian axes x, y, z that are parallel to the width, height, and length of the resonant cavity, respectively. For its part, v is the phase speed.

That is, using equation (1) resonance frequency values can be obtained for infinite “modes”. Each mode is a spatial arrangement specific to the electrical and magnetic fields within the resonator.

In said equation (1), n is the number of wave semi-cycles entering dimension x (width of the resonant cavity), m is the number of wave semi-cycles entering the y dimension (height of the resonant cavity), and/is the number of wave semi-cycles entering dimension z (length of the resonant cavity). In order to maximize the interaction of the finger and antenna on the energy of the excited wave, it is beneficial to place both in the exact positions of those maximums.

By means of some software to simulate electromagnetic problems, such as CST Studio or ANSOFT, the responses can be observed so as to determine the reflection coefficient parameter of the input, known as S11, which provides information on resonance as a function of changes in dielectric constant produced as a result of a variation of the dielectric constant due to the presence of glucose in blood.

For a constant frequency wave, switching from one medium to another with different μ (permeability) or ε (permittivity) produces a change in wavelength. This is the reason why the materials inside the resonator change the resonance frequency. The resonance condition is only met when the wavelength of the incoming signal (which depends on its frequency, and the characteristics of the medium) matches the resonance wavelength (which depends on dimensions). For example, the resonance frequency of one of the modes can be maintained by modifying the permittivity of the materials inside the cavity, as long as the cavity dimensions are changed accordingly.

Therefore, placing elements inside the resonator modifies the arrangement of stationary wave patterns, and resonance frequencies. This is the result of the discontinuity between materials and their electromagnetic properties.

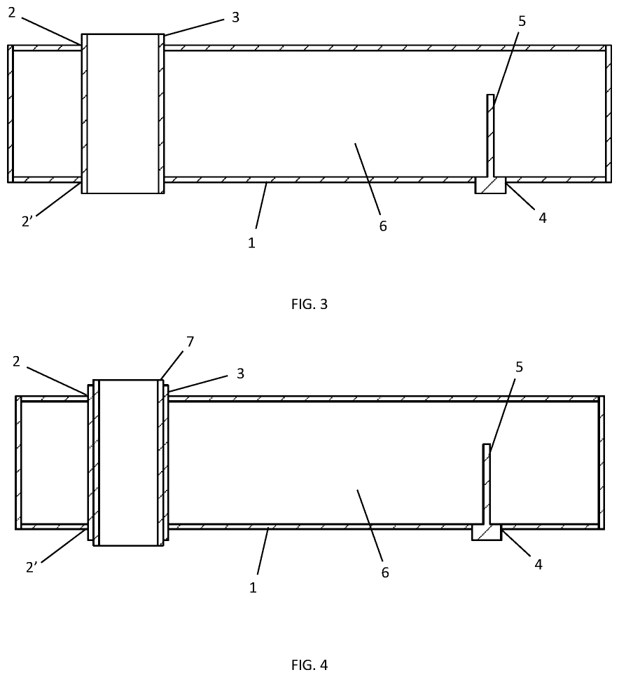

With reference to FIG. 3, it shows a cross-sectional side view of the resonant cavity of FIG. 2, where resonant cavity 1 is shown comprising holes 2 and 2′, mold 3, a hole 4 so that antenna 5 can be inserted, and a dielectric material 6.

Mold 3 is preferably made of rigid plastic material and comprises a hollow cylinder, preferably with a 1 mm wall thickness with holes at its longitudinal ends. Mold 3 can be tailored to a user's finger by using, for example, 3D printing to fit the finger shape and facilitate finger immobilization. However, mold 3 does not need to replicate exactly the shape of the user's finger, but its inner surface may have a slightly conical shape, not shown in FIG. 3, so that the finger “fills” the mold taking advantage of the elasticity of the structure of the human finger, thus achieving a better finger immobilization. To this end, a set of molds can be provided with small variations in their conicity and different sizes, so that the user can find a suitable one. Finger immobilization seeks to improve the repeatability of the measurement on the basis that the mold volume, which is practically constant by rigidity of the mold, is filled with the finger in the same way in each measurement, taking advantage of the flexibility of human tissue. Mold 3 is positioned upright passing through holes 2 and 2′ allowing to vary the vertical position of the finger so as to obtain the best response in frequency of the variation of parameter S11 of the finger-resonator assembly.

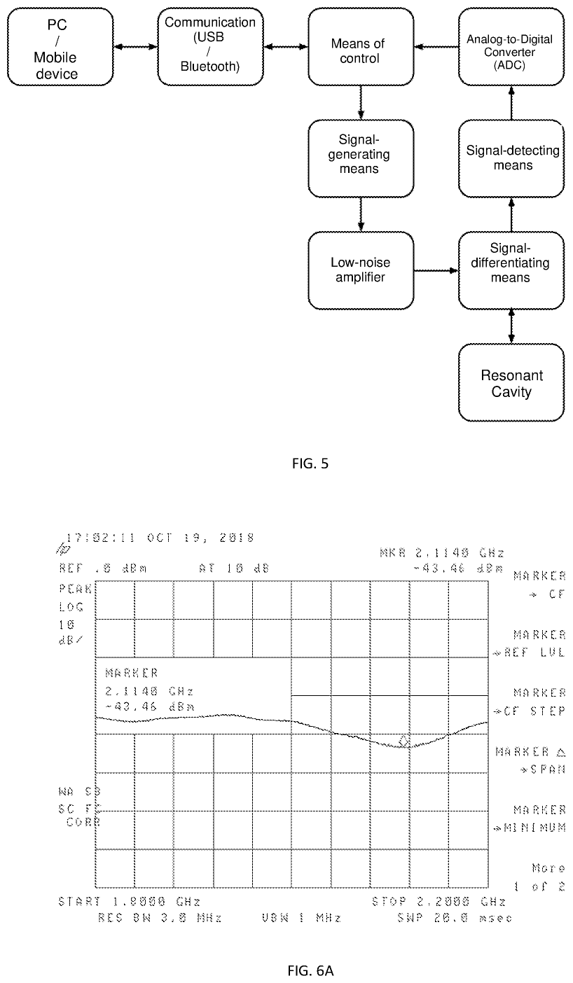

Antenna 5 generates an electric field due to microwave signals transmitted to resonant cavity 1 as it is connected to a signal-differentiating means and this, in turn, to a radiofrequency signal-generating means (not shown) capable of generating and scanning signals of microwaves in the frequency range of 0.5 GHz to 3 GHz. When a patient's finger under study is inserted into mold 3 it is possible to determine the disturbance in the electrical conditions of resonant cavity 1 generated by the introduction of the finger. The signal-differentiating means can be selected from the group comprising a bidirectional coupler, a directional coupler, and a circulator. In the case of the circulator, it must be specially designed in order to separate the reflected wave. In a preferred embodiment, the signal-differentiating means is a bidirectional coupler (not shown). This signal-differentiating means is essential for measuring the S11 response of the finger-resonator assembly which gives information about the present glycaemia level, since it separates the incident wave from the reflected wave allowing the measurement of the parameter S11 module. This signal-differentiating means is connected to the signal-generating means and also connected to a signal-detecting means (not shown) which synthesizes a signal proportional to the required measured amplitude and obtains in its frequency scan measurement the frequency response of the measuring device of the present invention. The signal-generating means and signal-detecting means can both be arranged, for example, by means of a spectrum analyzer (not shown), for example, the HP-8594E model produced by Hewlett-Packard or can be arranged separately by means of corresponding electronics. In addition, the measuring device has at least one means of control, such as, by way of example, a microprocessor or a programmable gates array, connected to the signal-detecting means and to the signal-generating means, and which controls the generation of signals and the reception of signals from the signal-detecting means. That is, it controls the operations of the measuring device, taking care of controlling the signal scan of the signal-generating means and acquiring amplitude signals from the signal-detecting means. In addition, it is in communication with either a PC or a mobile device, via USB or Bluetooth, so that all measured information can be processed to obtain the patient's glycaemia values and stored in order to keep a historical record of the measurements. In an embodiment, a low-noise amplifier (not shown) can be coupled between the signal-differentiating means and the radiofrequency signal-generating means so as to provide higher gain and frequency range levels. FIG. 5 shows in a schematic way how the various mentioned components interact with each other.

With reference again to FIG. 3, in order to maximize the influence of the finger on the response, and thus allow to detect the frequency changes caused by different levels of blood glucose, the finger should be inserted in a place where the resonator has an intense field distribution. Since the accommodation of electromagnetic fields occurs in an integer number of wavelength semi-cycles, which are in the form of a sinusoidal-type function, it is desirable that in this distribution of sinusoidal semi-cycles, the finger is located at the geometric point of maximum value, thus altering the resonance as intensely as possible. Therefore, mold 3, for finger placement and holding, and antenna 5 are located so that the electric field maximizes its intensity over the finger geometry, thus increasing the effect of dielectric finger changes due to changes in glycaemia. In this way the most intense glycemic patterns are reflected in the values of the resonance frequencies, which is what is sought to increase sensor sensitivity.

Dielectric material 6 is preferably an epoxy resin because its relative dielectric constant ε_{R} is approximately equal to 4.3 what leads to the wavelength inside the resonator to be in the order of the finger diameter, allowing that in the sinusoidal semi-cycles distribution the finger is immersed in the region of maximum amplitude value of this geometric wave distribution. This maximizes the influence of the finger on the resonance frequency, as well as the variation in frequency with changes in glycaemia.

Performing various simulations using CST Studio or ANSOFT with different dimensions of width, height and length of resonant cavity 1, it was found that in order to achieve an adequate isolation between resonance modes it is convenient that height b be equal to half the width a of resonant cavity 1,

$$i . e . b = \frac{a}{2} .$$

In addition, the use of epoxy resin as a dielectric material allows a design of acceptable dimensions for resonant cavity 1 such as a=50 mm, b=25 mm, L=110 mm. For these dimensions it is possible to obtain an adequate resonance mode which resonates in frequencies around 2 GHz which allows the use of commercially available electronics to implement the metering device of the present invention. As noted above, these dimensions change when the filling material has another relative dielectric constant different than the one used.

In a preferred embodiment, a resonance mode is adopted where the electric field is distributed in two sinusoidal semi-cycles along the length L of the resonator. Each semi-cycle has a maximum electric field. The design is based in principle on housing the two objects of interest, the mold-finger assembly and the antenna, in those maximum electrical field positions. To his end, hole 4 to insert antenna 5 is at a distance equal to a quarter of the length L of resonant cavity 1 relative to one end, and holes 2 and 2′ for inserting mold 3 are at a distance equal to a quarter of the length L of resonant cavity 1 relative the other end, so that hole 4 for inserting antenna 5 and holes 2 and 2′ for inserting mold 3 are separated from each other by a distance equal to half the length L of resonant cavity 1.

In an even more preferred embodiment, hole 4 for inserting antenna 5 is at a distance approximately equal to one fifth of the length L of resonant cavity 1 relative to one end and holes 2 and 2′ for inserting mold 3 are at a distance approximately equal to one fifth of the length L of resonant cavity 1 relative to the other end, so that hole 4 for inserting antenna 5 and holes 2 and 2′ for inserting mold 3 are separated from each other by a distance approximately equal to three-fifths of the length L of resonant cavity 1.

In a more preferred embodiment, the distance of mold 3 from antenna 5 is half a wavelength, i.e. at maximum electric field in order to maximize system resolution. In yet another preferred embodiment, antenna 5 is located at a distance of one quarter of a wavelength from one end of resonant cavity 1 and mold 3 for finger location and hold is located at a half wavelength distance from antenna 5 so as to optimize sensor resolution.

In a more preferred embodiment of the present invention, considering the dimensions of resonant cavity 1 mentioned above, that is, a=50 mm, b=25 mm, L=110 mm, the optimal location of mold 3 is at a distance of 21 mm from one end of resonant cavity 1 and the optimal location of antenna 5 is at a distance of 22 mm from the other end of resonant cavity 1.

With reference to FIG. 4, it shows a cross-sectional side view of a preferred embodiment of the present invention, where mold 3 comprises an additional mold 7 of flexible plastic material for holding the user's finger, which is also hollow and presents holes at its ends. In this way mold 3 is fixed to resonant cavity 1 and it is said additional mold 7 which takes care of the finger location and hold. In this case, mold 7 can be tailored to the user's finger or may have a slightly conical inner surface (not shown) and is made of flexible plastic material so that the finger adapts much better to mold 7 regardless of the time of day and the volumetric expansion that the finger or mold 3 might have.

In an even more preferred embodiment of the present invention, the mold comprises an optical distance sensor (not shown) to determine and control the penetration level of the finger, that is, how much the finger entered in mold 7. Thus, the distance value between the optical sensor and the finger is recorded and the repeatability of this distance for subsequent measurements is achieved. In addition, a measurement can be considered correct, or having a minimal error, when the finger entered mold 7 correctly and the distance to the optical sensor is equal or similar to that obtained during the calibration process, which is described below. El optical sensor is located on the outside and on the bottom face of resonant cavity 1, making hole 2′ necessary.

With reference to FIG. 5 again, it shows schematically how the different components that can form, by way of example, the measuring device of the present invention interact with each other. In addition to the above, if a bidirectional coupler is used as a signal-differentiating means, it is based on microstrip technology. In particular, the bidirectional coupler design is based on coplanar techniques using FR4 as support material. In addition, the at least one means of control can receive, by means of an analog-to-digital converter, the signals captured by the signal-detecting means.

By means of at least one means of control and the means of generating radiofrequency signals, a frequency scan is made and microwave, preferably in the range of 0.5 to 3 GHz, are sent to the resonant cavity by means of the signal-differentiating means and the antenna contained in said cavity and then by means of the signal-differentiating means, the antenna and the signal-detecting means capturing and obtaining the frequency response of the reflected waves. Said signal-differentiating means, as described above, makes it possible to differentiate microwaves sent to the resonant cavity from the waves reflected in the resonant cavity. From the obtained frequency response, two parameters are obtained that correspond to the resonance frequency and bandwidth at the resonance frequency, in which is the most significant information that allows to relate the frequency with glycaemia. The frequency response of the measuring device can be processed in various ways as described below in different embodiments.

In an embodiment of the present invention, a neural network is implemented, with which, by inputting the parameters of resonance frequency and bandwidth and parameters specific to the patient such as sex, height, weight, age, cholesterol level, hematocrits, among others, the value of final glycaemia is obtained. Such a neural network can be developed using different network topologies, such as feedforward, and different software, such as Python, and can be implemented on different devices such as cell phones, or even in the means of control.

This neural network works based on three stages. The first stage defines the morphology and main characteristics of the network. Once defined, the second stage proceeds which is a training session, in which a series of input-output values (in this case frequency—glycaemia) are delivered to the network and errors are corrected iteratively. The network is said to be trained when the error between known and predicted values by the neural network is less than an initially defined threshold. Finally, the third stage is the use stage, in which when inputting the neural network with the frequency values obtained by the measuring device, the network can predict the corresponding glycaemia. In other words, the implemented network functions as a predictor, responsible for translating the measured parameters (frequency response, bandwidth, resonance frequency, etc.) into a glycaemia value.

In another embodiment of the present invention, a polynomial is applied that allows converting frequencies into glycaemia values. This mechanism has the advantage that it is simple to implement, as it requires only a simple mathematical procedure to obtain the glycaemia value.

In another more preferred embodiment of the present invention, a joint system is implemented, joining characteristics of the two systems mentioned above. Given the characteristics of the two methods and their advantages, a more complex system is implemented, which allows reducing the error while maintaining a simple implementation. In addition, this system includes another parameter associated with the validity of the measurement, with which, observing the characteristics of the obtained curves, it may be determined whether it was a correct or erroneous measurement. On the other hand, training can be performed dynamically, and increase system accuracy as the number of samples increases.

### Calibration of the Measuring Device

The designed resonant cavity is characterized by allowing the existence of electromagnetic waves inside, preferably with specific frequencies that are dependent on their dimensions. When analyzing the reflected wave, at frequencies from 0.5 to 3 GHz, the amount of return energy is minimal, so resonant frequencies can be observed as peak values in the transfer. It is this resonance that is affected when the finger enters the mold, and shifts to another frequency value. For the proper functioning of the measuring device, it is necessary that the influence on the response of that resonance caused by finger insertion is as intense as possible, so that changes in blood glucose level are reflected as clearly as possible as changes in the resonance frequency value. Due to the strong influence of various parameters of this resonance, which depend on the individual characteristics of each volunteer, the device requires an individual calibration, which is performed with the volunteer who will perform the clinical test. The calibration process, described below, seeks to maximize the finger effect on the frequency response of the resonator-finger system.

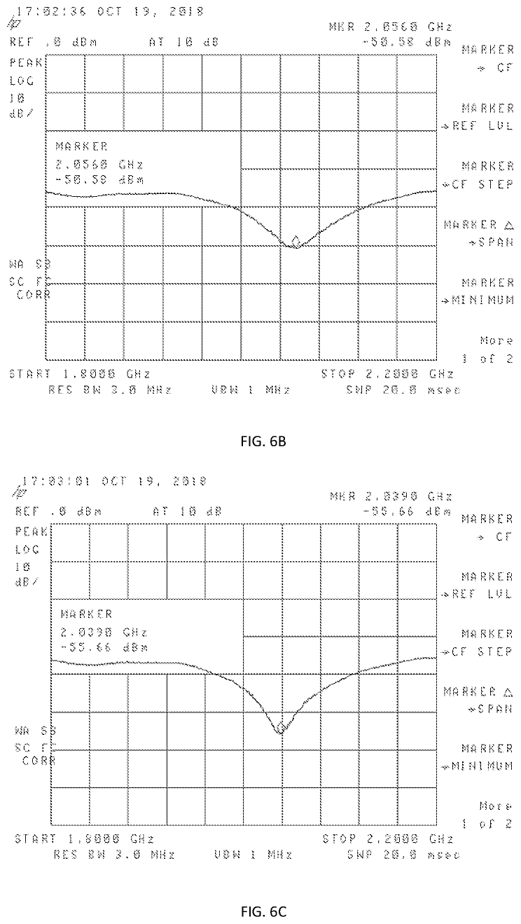

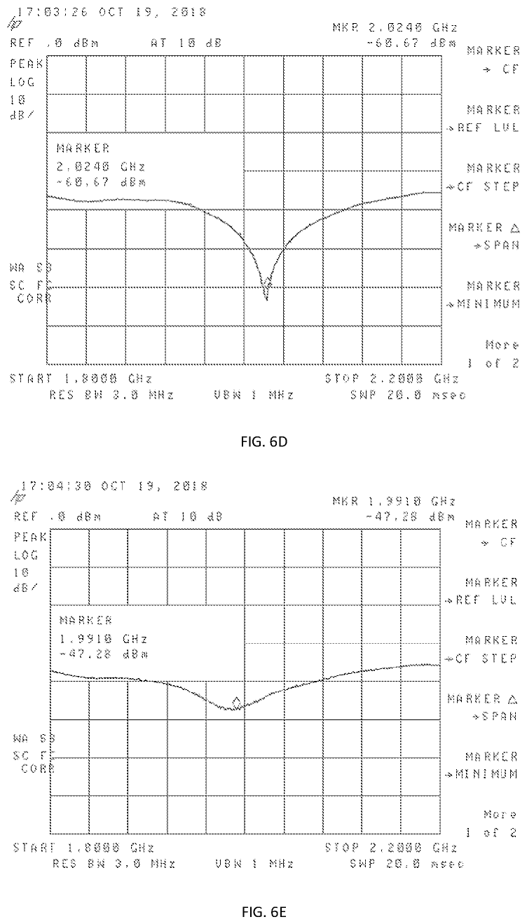

FIGS. 6A to 6E show the frequency response of the measuring device of the present invention after a frequency scan using a spectrum analyzer and a bidirectional coupler in order to measure the response of the S 11 parameter. FIG. 6A corresponds to the frequency response of the measuring device without the introduction of a user's finger into the mold of the device resonant cavity. FIGS. 6B to 6E correspond to the frequency response of the measuring device as a user's finger enters the mold of the resonant cavity, where in FIG. 6B the finger being inserted a certain depth. In FIG. 6C the finger is inserted at a depth greater than the depth corresponding to FIG. 6B. In FIG. 6D the finger is inserted at a depth greater than the depth corresponding to FIG. 6C. Finally, in FIG. 6E the finger is inserted at a depth greater than the depth corresponding to FIG. 6D.

The frequency response in each of these FIGS. 6A to 6E is shown in the frequency range from 1.8 GHz to 2.2 GHz, as it is known that this is the range of interest, where the response is optimal.

It can be seen in said FIGS. 6A to 6E that when the finger enters and as it is entering the resonator, the peak (negative) amplitude of resonance increases in magnitude and shifts decreasing in frequency until it is at its optimum point which corresponds to FIG. 6D which has a peak amplitude of −60.67 dBm (decibel-milliwatt) and a resonance frequency of 2.0240 GHz. When continuing entering the finger, past the optimal point, the resonance continues to decrease in frequency but begins to decrease in magnitude as can be seen in FIG. 6E whose peak amplitude is −47.28 dBm and its resonance frequency of 1.9910 GHz. The reason why a good calibration is of great importance, is that the higher the quality factor of said resonance (the negative peak is more abrupt), the frequency variation produced by glycaemia is greater, and hence a measurement can be obtained with a lower error. Finally, through the results obtained from the different positions of the finger, it is possible to identify which level of insertion of the user's finger leads to observing the most intense response so that, as mentioned above, changes in blood glucose level are reflected as clearly as possible as changes in the value of the resonance frequency.

For the calibration of the measuring device of the present invention, the frequency response obtained by the non-invasive measuring device is contrasted with blood glucose concentration values obtained in the laboratory following the “gold standard” or some other invasive glucose measurement device that serves as a pattern.

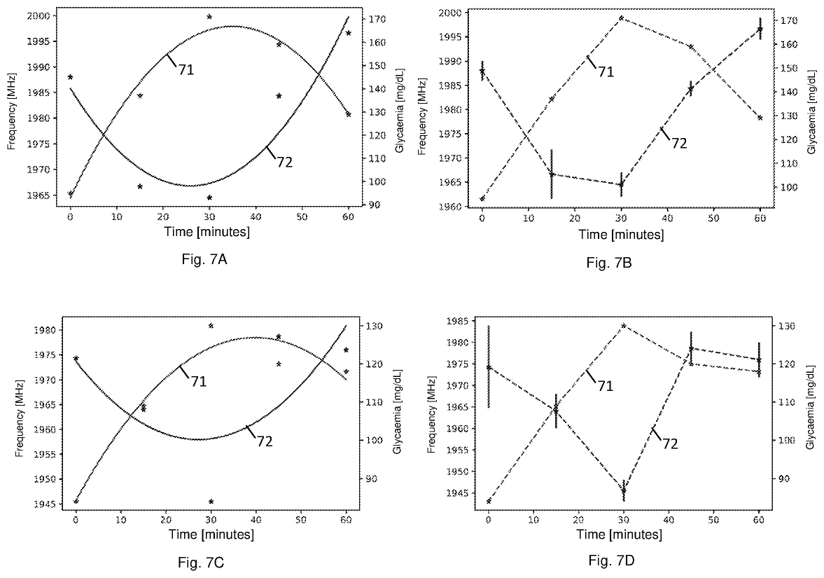

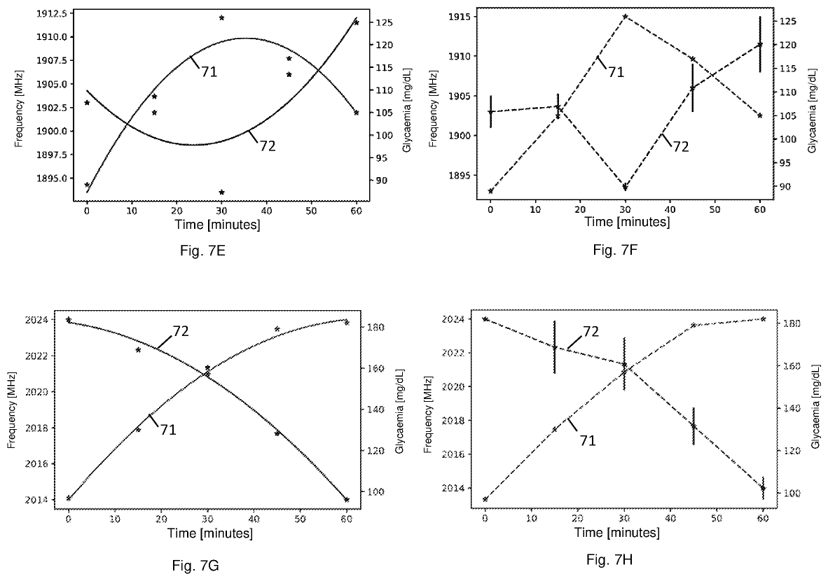

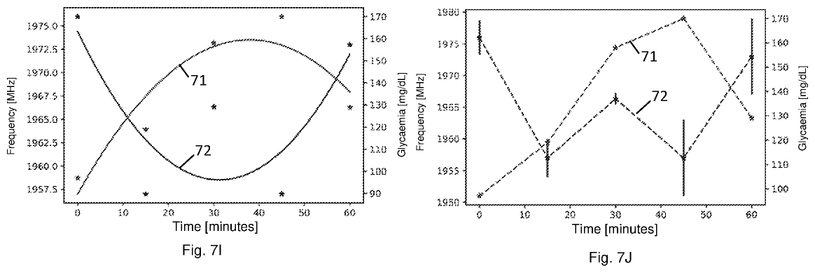

As only an example of calibration and the accuracy of the measuring device, clinical tests were performed on healthy volunteers (non-diabetic, i.e. baseline glycaemia values between 70 mg/dL and 110 mg/dL) consisting of a glycaemia overload, in which each of the volunteers, after between eight and ten hours of fasting, ingested 75 g of glucose powder dissolved in water and their glycaemia levels were observed every 15 minutes. A moment before ingesting glucose, at 15 minutes, 30 minutes, 45 minutes, and 60 minutes after ingesting glucose, measurements were made with the non-invasive measuring device and blood samples were taken for laboratory testing. In FIGS. 7A to 7J, the data obtained through clinical tests can be seen in the form of graphs. All represent, as a function of time, the evolution observed in volunteers, both regarding their blood glucose concentration, curves 71, and the resonance frequency measured through the resonant cavity of the measuring device, curves 72. These graphs in FIGS. 7A to 7J correspond row by row to the same person. On the left, the graph shows a approximation by quadratic regression of the obtained data, and on the right, for each case, the same data are shown joined by linear sections in dotted line.

For such graphs, blood glucose is extracted from a laboratory test following the “golden standard”, which is adopted as a rule for calibration, and therefore error-free. Instead, the resonance frequency has slight changes in a single moment of measurement, resulting from errors in the position of the finger. The average measured frequency value along with its error bars are included in the frequency data of the right-side charts (in dashed-line), for each sampling moment, symbolizing the range of variation that was experienced during the test when changing the position of the finger. Finally, adjacent points are joined together, as linear interpolation, to favor visualization.

On the other hand, the charts on the left side include the same sampled points, for both variables of interest, but overlapping with trend curves (in continuous line) calculated by quadratic regression, following the criterion of least squares. These are the second-order polynomials that best fit the data, in each case, and that would allow predicting, with minimal error (understood as the smallest possible average error, for the regression order used), the glycaemia and frequency values that would be observed at every moment of time. These charts are used to qualitatively identify trends, or behavior patterns, in the data, and to corroborate the measurement capability of the measuring device. Not all charts are displayed with the same scale, but expanded information is displayed so that, in each case, the curves occupy the widest possible range.

The graphs shown in FIGS. 7A to 7J show that when glycaemia curves have an increasing behavior, frequency curves have a decreasing behavior, and when glycaemia has a decreasing behavior, frequency curves have an increasing behavior.

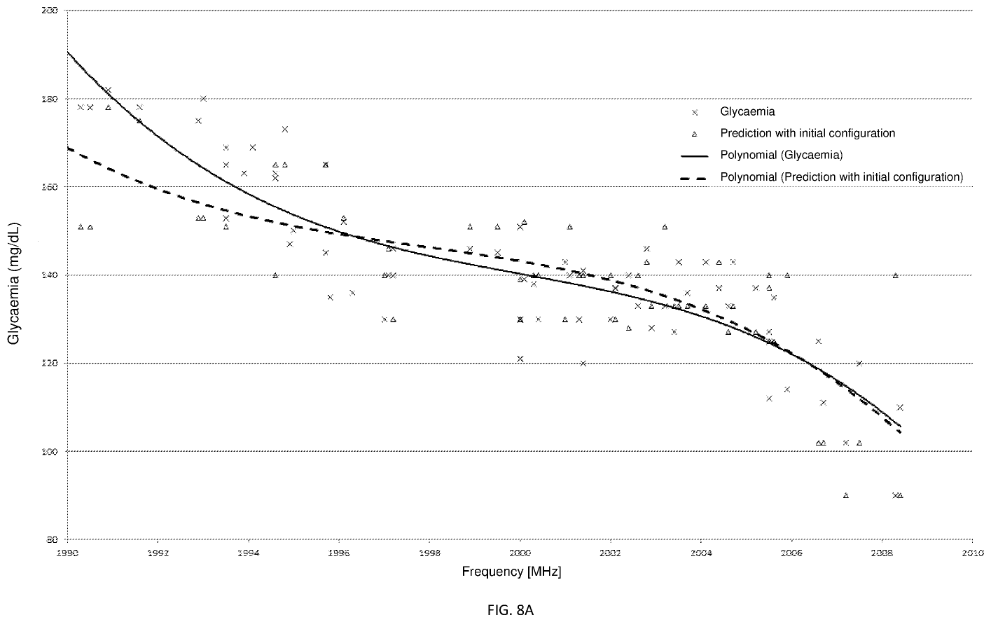

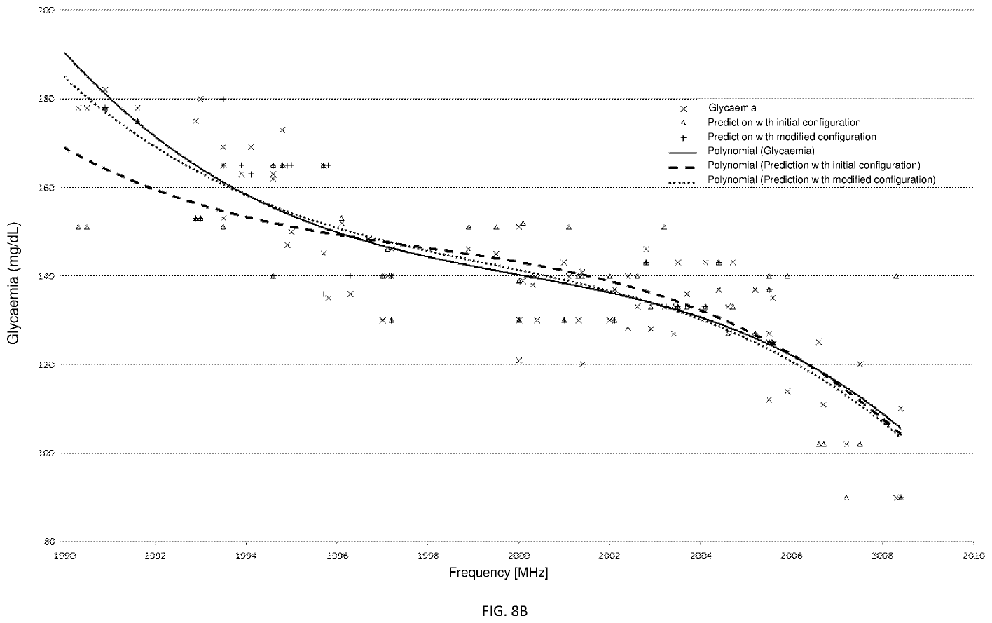

For metering device users being able to use it, they initially have to calibrate the device. To do this, they must take samples day by day with a conventional glucometer (normally), i.e. invasive, and compare the measurements of the glucometer with those obtained by the non-invasive measuring device. An example of calibration can be seen in FIGS. 8A and 8B, where the non-invasive metering device was used, which already had a first calibration so that it could translate the frequency response into the user's glycaemia values, employing an automatically self-training neural network, by comparing the glycaemia values predicted by the invasive and non-invasive glucometers, and adding corrections to the non-invasive estimate on a daily basis. The goal of FIGS. 8A and 8B is to demonstrate that, as the number of samples increases, the device is capable to obtain increasingly reliable glycaemia information. The invasive glucometer used has a ±15% error and 25 samples were employed. For both FIGS. 8A and 8B, the abscise axis represents the resonance frequency of the resonant cavity of the measuring device in MHz and the ordinate axis represents glycaemia at a concentration of mg of glucose per dL of blood.

FIG. 8A shows initially in a first stage of the process typically performed by neural networks, the discrepancy between the data corresponding to invasive measurements, taken as a pattern, represented by crosses (x) and approximated by a continuous line curve, with the data corresponding to non-invasive predictions represented by triangles and (A) approximated by a dashed-line curve. However, once a larger amount of data was obtained, the neural network went through stages of self-training, obtaining new predictions represented by the plus sign (+) and approximated by a dotted curve, which can be seen in FIG. 8B. It can also be observed that the dotted curve is closer to the continuous line curve than the dashed-line curve, as expected, so increasing the number of measurements, the error greatly decreases. In this way, a polynomial equation of the dotted curve that characterizes glycaemia in function of resonance frequency can be obtained. Such an equation in this case would be

glyc(_{f})=−0.0141921.f³+85.1084179.f²−170129.99818.f+113364033.3241

where f is the resonance frequency and glyc_{(f)} is the glycaemia value corresponding to said resonance frequency value.

Once the approximation curve is achieved, the values thereof can be replaced either through a PC or mobile device and the conversion between frequency and glycaemia directly made with the aforementioned curve. In other words, the non-invasive glycaemia prediction made with the noninvasive metering device would be sufficiently accurate, and it would not be required to continue to train the device with the invasive glucometer. The non-invasive metering device would be independent from now on, having an associated error that would tend to that of the invasive glucometer since it was based thereon for calibration, i.e. ±15%. However, as a person skilled in the art will appreciate, if the data provided to the measuring device of the present invention for training the neural network came from a gold standard, they would bring the non-invasive prediction of the non-invasive measuring device closer to actual glycaemia.

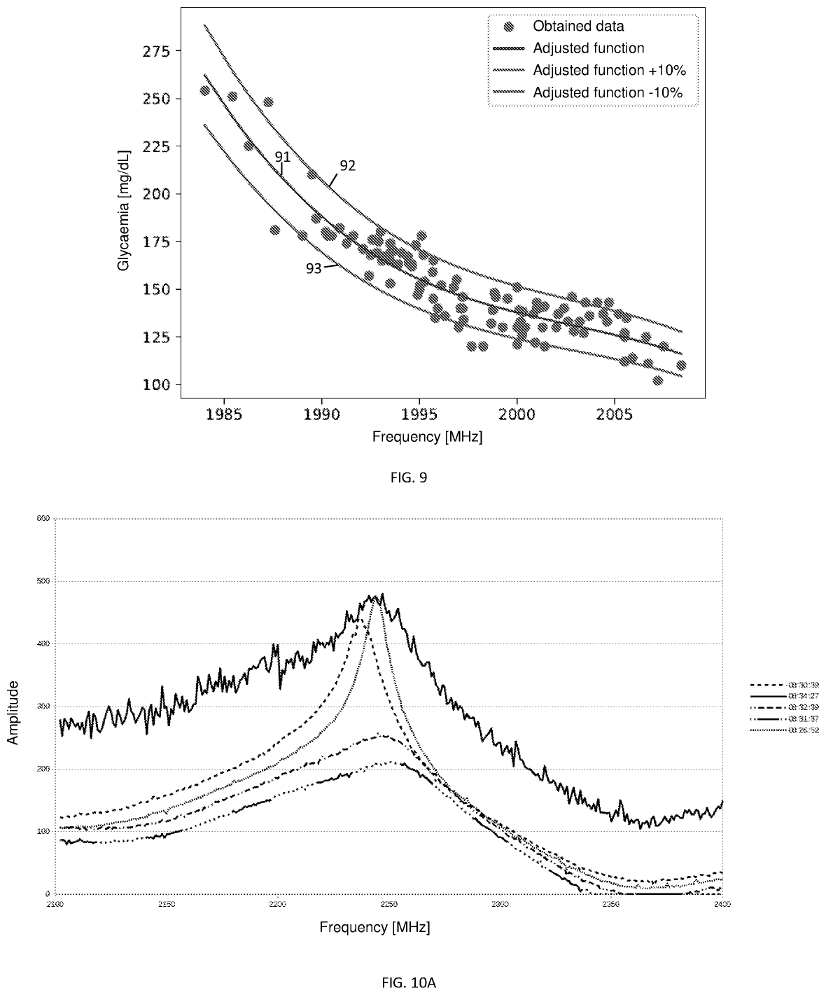

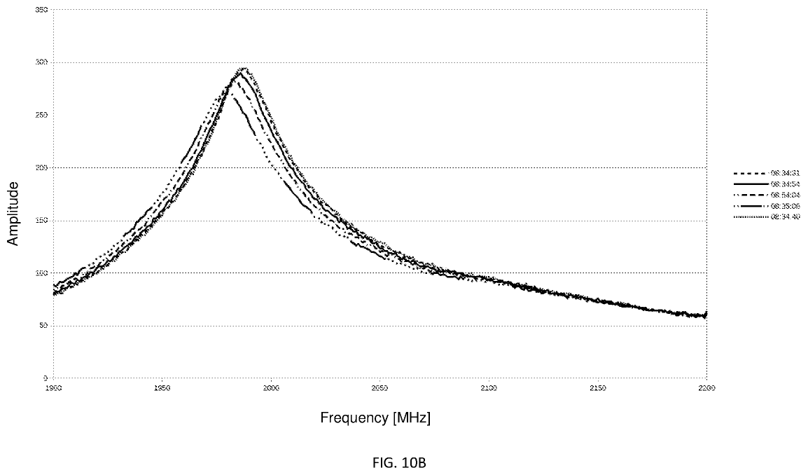

Finally, FIG. 9 shows a graph of glycemia vs. resonance frequency as an example. Continuous curve 91 corresponds to an order 3 least squares regression, and curves 92 and 93 corresponding to a ±10% error of curve 91 were added so as to visualize the dispersion in the values. Said curve 91 is the calibration curve of the non-invasive metering device of the present invention obtained for a volunteer with diabetes with which he can predict the glycaemia value from the non-invasive metering device. Said curve was obtained by the volunteer, as explained above, using as a pattern an invasive blood glucose meter, which was an Accu-Chek Performa glucometer and has an error of ±20%. Thus, the non-invasive metering device would tend to have a ±20% error.

### Comparative Results

The following are the improvements of the device of the present invention compared to the AR 104766 A1 patent application device. Unlike the AR 104766 A1 patent application device, the device of the present invention is not susceptible to the electromagnetic fields present in the environment because the hollow prismatic resonant cavity with rectangular cross section has a closed arrangement so as to avoid interference that could be caused by such electromagnetic fields. In addition, FIGS. 10A and 10B, show two clinical tests, corresponding to the same volunteer, and carried out with the two compared sensor topologies, that is, with the open coplanar structure of patent application AR 104766 A1, FIG. 10A, and with the resonant cavity of closed arrangement corresponding to the device of the present invention, FIG. 10B. In these FIGS. 10A and 10B it can be observed how, in multiple measurements made on a finger of a user with a given glycaemia level, at different but close time moments, which should have the same resonant amplitude and frequency in each of the measurements, the open coplanar arrangement exhibited a significant variability in successive measurements, both in resonance frequency (by effect of differences in pressure and position), and in the noise added to the curve obtained after measurement by random interfering sources, thus affecting the measurement repeatability. In other words, FIG. 10A shows the problem of repeatability of the open coplanar structure of patent application AR 104766 A1, since repeating the measurement generated a lot of error and curves not correlated with each other.

On the other hand, in the case of the measurement with the resonant cavity of closed arrangement of the present invention, the repeatability turned out to be much better, presenting minimal discrepancies between different measurements, as can be seen in FIG. 10B. It should be noted that once the user of the non-invasive metering device is trained for the use thereof, the obtained measurement curves would be practically the same.

1. Non-invasive measuring device for measuring blood glucose concentrations on a user's finger, based on microwave signals sensitive to differences in electrical permittivity caused by changes in blood glucose concentration, wherein the device comprises:
a hollow prismatic resonant cavity, which has a width, height and length, with rectangular cross-section defined by the width and the height of the resonant cavity, being the longitudinal central axis of the resonant cavity in a horizontal position, where the resonant cavity is filled with a dielectric material, and where the resonant cavity comprises:
two longitudinally opposite faces defined by the width and the height of the resonant cavity that define longitudinal ends of the resonant cavity, two laterally opposite faces defined by the length and the height of the resonant cavity, and two vertically opposite faces defined by the width and the length of the resonant cavity;
an antenna inside the resonant cavity, with the capability to transmit a microwave signal to the blood, with the antenna being inserted into a hole located on one of the laterally opposite or vertically opposite faces;
a mold for location and hold of the user's finger inside the resonant cavity, with the mold being inserted into a hole located on at least one of the laterally opposite faces or in a hole located on at least one of the vertically opposite faces;
a signal-generating means capable of generating microwave signals connected to the antenna through a signal-differentiating means;
a signal-detecting means connected to the antenna through the signal differentiating means; and
at least one means of control, connected to the signal-generating means and to the signal-detecting means, which controls signal generation from the signal-generating means and the reception of signals from the signal-detecting means.

2. The measuring device according to claim 1, wherein the resonant cavity is obtained from a metallic hollow prismatic profile.

3. The measuring device according to claim 2, wherein the hollow prismatic profile is made of bronze.

4. The measuring device according to claim 1, wherein the signal-generating means generates signals in microwaves in the range of 0.5-3 GHz.

5. The measuring device according to claim 1, wherein the dielectric material is epoxy resin that has a relative dielectric constant ER approximately equal to 43.

6. The measuring device according to claim 1, wherein the signal-differentiating means is selected from the group comprising a bidirectional coupler, a directional coupler and a circulator.

7. The measuring device according to claim 1, wherein the at least one means of control comprises a means of control connected to the signal-detecting means and which controls the reception of signals from the signal-detecting means, and a means of control connected to the signal-generating means and which controls the generation of signals.

8. The measuring device according to claim 1, wherein the at least one means of control is selected from the group comprising a microprocessor and a programmable gate array.

9. The measuring device according to claim 1, wherein both laterally opposite faces each comprises a hole for mold insertion so that both holes are concentric to each other.

10. The measuring device according to claim 1, wherein both vertically opposite faces each comprises a hole for mold insertion so that both holes are concentric to each other.

11. The measuring device according to claim 1, wherein the antenna and the mold are parallel to each other.

12. The measuring device according to claim 1, wherein the antenna and the mold are located on the same face and aligned with the longitudinal center axis of the resonant cavity.

13. The measuring device according to claim 1, wherein the resonant cavity has a width equal to twice its height.

14. The measuring device according to claim 13, wherein the resonant cavity has a width of 50 mm, a height of 25 mm and a length of 110 mm.

15. The measuring device according to claim 14, wherein the optimal location of the mold is at a distance of 21 mm from one end of the resonant cavity and the optimal location of the antenna is at a distance of 22 mm from the other end of the resonant cavity.

16. The measuring device according to claim 1, wherein the antenna is at a distance equal to a quarter of the length of the resonant cavity relative to one end and the mold is at a distance equal to a quarter of the length of the resonant cavity relative to the other end, so that the antenna and mold are separated from each other by a distance equal to half the length of the resonant cavity.

17. The measuring device according to claim 1, wherein the antenna is at a distance approximately equal to one fifth of the length of the resonant cavity relative to one of the ends and the mold is at a distance approximately equal to one fifth of the length of the resonant cavity relative to the other end, so that the antenna and the mold are separated from each other by a distance approximately equal to three-fifths of the length of the resonant cavity.

18. The measuring device according to claim 1, wherein the position of the mold relative to the position of the antenna is half the wavelength of the microwave used.

19. The measuring device according to claim 1, wherein the antenna is located at a distance of one quarter of the wavelength of the used microwave from one end of the resonant cavity and the mold is located at a distance of half a wavelength of the used microwave from the antenna.

20. The measuring device according to claim 1, wherein the mold for finger location and hold is made of rigid plastic material.

21. The measuring device according to claim 20, wherein the device comprises an additional flexible plastic material mold for holding the user's finger, located inside the rigid plastic material mold.

22. The measuring device according to claim 21, wherein the rigid plastic mold has an inner threading for fastening the flexible plastic material mold.

23. The measuring device according to claim 21, wherein the flexible plastic material mold comprises an optical distance sensor for controlling that the finger penetration level is suitable for measurement.

24. The measuring device according to claim 1, wherein the device measures the reflection coefficient of the resonant cavity input.
